(19) United States
(12) Patent Application Publication
(10) Pub. No.: US 2024/0389840 A1
(43) Pub. Date: Nov. 28, 2024

(54) ENDOSCOPY SYSTEM & ELEMENTS THEREOF

(71) Applicant: I.Q. ENDOSCOPES LTD, Chepstow, Gwent (GB)

(72) Inventors: Andrew MILLER, Chepstow, Gwent (GB); Patrick WARD-BOOTH, Chepstow, Gwent (GB); Daniel Peterson GODFREY, Cambridge (GB)

(21) Appl. No.: 18/691,578

(22) PCT Filed: Sep. 6, 2022
(86) PCT No.: PCT/EP2022/074742

§ 371 Date: Mar. 13, 2024

(30) Foreign Application Priority Data
Sep. 14, 2021 (GB) 2113108.1

(51) Int. Cl. A61B 1/008

(52) U.S. Cl. CPC A61B 1/008

(57) ABSTRACT

Various embodiments provide a bendable member for an imaging endoscope and an imaging endoscope incorporating said bendable member. The bendable member comprises a series of constructional elements arranged longitudinally in series along the bendable member and formed integrally with a plurality of living hinge elements that permit bending between adjacent constructional elements, wherein, when the bendable member is straight, a first living hinge element of the plurality of living hinge elements has a different bending stiffness to a second living hinge element of the plurality of living hinge elements.

This application claims priority from GB2113108.1 filed 14 Sep. 2021, the contents and elements of which are herein incorporated by reference for all purposes.

### FIELD OF THE INVENTION

The present invention relates to imaging endoscope systems, elements thereof, methods for the assembly of such systems and to methods of operating such systems.

### BACKGROUND

It is known that early detection of disease may be assisted by endoscopic examination of internal structures such as the alimentary canals and airways, e. g., the oesophagus, lungs, colon, uterus, and other organ systems. Endoscopic examination may be carried out using an imaging endoscope. Typically, an imaging endoscope has a flexible tube providing a light guide that guides illumination light from an external light source located proximally of the flexible tube to a distal tip of the flexible tube. The illumination light thereby illuminates the tissue to be examined. The distal tip typically also includes an objective lens to gather light from the tissue being examined. The flexible tube provides an imaging light guide to carry the light away from the distal tip and to a camera at the proximal end of the flexible tube. It is also known for the imaging light guide to be dispensed with and replaced with an imaging camera chip at the distal tip. In this case, the signal from the imaging camera chip is conducted along electrical wires in the flexible tube. Based on either approach, an image is produced for display to the imaging endoscope operator.

It is also known, in addition to the imaging function described above, for imaging endoscopes to provide further functionality. For example, it is known for the distal tip to be equipped with a port for dispensing air (or more generally, insufflation gas) to inflate the internal structure being examined, a port for dispensing irrigation liquid such as water or saline, and a port for a medical tool such as biopsy forceps. To allow such services to be provided, the flexible tube typically provides suitable lumens dedicated to the provision of these services.

Although some endoscopic procedures can be carried out with a rigid insertion tube, for many procedures it is preferred that the insertion tube is flexible. This allows the insertion tube to be located along tortuous and often complex paths within the body. To allow adequate steering of the flexible tube within the body, it is known to provide the distal tip of the insertion tube with means for deflecting the distal tip, and therefore with means to steer the distal tip. A typical approach to this is to provide control cables, extending along the flexible tube from a control handle at a proximal end of the flexible tube and anchored to the distal tip. The control handle has control knobs to allow movement of the control wires and consequently steering of the distal tip.

Various arrangements to allow flexibility of the insertion section, and in particular the distal tip of the insertion section (sometimes referred to as a ‘steering section’) are known.

For example, U.S. Pat. No. 10,052,013B2 discloses an articulation joint for use in an endoscope. In one arrangement disclosed therein, the articulation joint body comprises a plurality of interconnecting segments. Each segment comprises a cylinder with an outer wall and a central lumen. The outer wall includes a number of hinge elements therein and a series of slots therethrough. A plurality of cable guide elements having a passage for control cables are inserted into the slots and two or more cables are threaded through the plurality of cable guide elements and tensioned to form the articulation joint body. In another arrangement disclosed therein, the articulation joint body is a tubular body comprising a cylinder with an outer wall and a central lumen. The outer wall includes a number of hinge elements therein and a series of slots therethrough. A plurality of annular rings are snap-fitted around the circumference of the tubular body at spaced intervals. Each annular ring has an outer circumference with a first end and a second end and a space therebetween. Also included in each annular ring is at least one pair of inwardly extending cable guide loops adapted to be inserted into the slots in the outer wall of the tubular body. Two or more cables are threaded through the plurality of cable guide loops and tensioned to form the articulation joint body.

EP2617350 B1 describes multiple different possible configurations of a bendable insertion section and steering section of an imaging endoscopy system.

However, many existing articulating members for endoscopy systems are complex to manufacture or assemble, or relatively high cost to produce, thereby making them unsuitable or undesirable for use in low cost endoscopy systems, such as single use endoscopy systems.

The present invention has been devised in light of the above considerations.

### SUMMARY OF THE INVENTION

The present inventors have realized a number of innovations which can reduce the overall cost and complexity of manufacture of an insertion section of an imaging endoscopy system, whilst providing suitable or improved performance in comparison to conventional endoscopes.

The primary innovations referred to herein are referred to as ‘Innovation A’ and ‘Innovation B’. In addition to these primary innovations, a number of further innovations are identified which may find application independently of Innovation A or Innovation B. These further innovations are indicated below as being further independent aspects of the present disclosure.

Whilst discussed separately, it will be understood that these innovations are nevertheless combinable, and accordingly, the invention includes the combination of the innovations, aspects of innovations and preferred features described except where such a combination is clearly impermissible or expressly avoided.

### Innovation A

In a first aspect of Innovation A, the present invention provides a bendable member for an imaging endoscope, said bendable member having a longitudinal axial direction, a circumferential direction and a radial direction, wherein the bendable member comprises:
a sheath portion comprising a series of sheath ring segments formed integrally with sheath living hinge elements, the sheath portion being bendable by bending of the sheath living hinge elements; and
a spine portion comprising a series of radially extending spacer segments formed integrally with spine living hinge elements, the spine portion being bendable by bending of the spine living hinge elements; wherein
the spine portion and the sheath portion are coaxially arranged such that one or more longitudinally extending conduits are defined between the spacer segments of the spine portion and the sheath ring segments of the sheath portion.

Providing such an arrangement, where the spine portion and the sheath portion are coaxially arranged such that one or more longitudinally extending conduits are defined between the spacer segments of the spine portion and the sheath ring segments of the sheath portion, allows for improved ease of manufacture of the bendable member in comparison to prior art systems. In particular, during assembly of the bendable member, the spine portion can be effectively used as a scaffold frame, onto which other components of the endoscope (control wires, electrical wires, fluid conduits etc.) can be disposed on or held by the spacer segments of the spine portion, with the sheath portion subsequently being arranged to cover the spine portion and the one or more other components of the endoscope. The one or more other components are then held within the longitudinally extending conduits defined between the spacer segments of the spine portion and the sheath ring segments of the sheath portion. This is a much faster assembly method than conventional methods which require the careful threading of control wires, electrical wires, fluid conduits etc. along the length of a bendable member of convention endoscopes.

Furthermore, because both the spine portion and the sheath portion are bendable by bending of living hinge elements, it is possible to provide a two-part component where the bending response of both the spine part and the sheath art is highly selectable (for example, to provide a similar bending response to an applied force, or to provide a different bending response to an applied force), in comparison to some known arrangements where the bendable member is constituted by an internal articulated jointed section comprising a series of ring links surrounded by an external sheath. This is because in an arrangement according to the present invention, the bending response of both the spine portion and the sheath portion can be selected at the point of manufacture by suitable selection of the location and form of the living hinge elements.

Accordingly, a bendable member according to the present invention can allow for greater customization of bending response in comparison to existing bendable members.

Each of the sheath portions and the spine portion is formed as a series of constructional elements (sheath ring segments for the sheath portion, or spacer segments for the sine portion) arranged longitudinally in series along the bendable member and formed integrally with a plurality of living hinge elements.

The term ‘living hinge element’ is used herein to define a flexible hinge integrally formed with, and preferably made from the same material as, the two segment pieces it connects. The living hinge elements of the sheath portion and/or the spine portion may be formed by local material removal (e.g. cutting) of the sheath portion and/or spine portion. Alternatively, the living hinge elements of the sheath portion and/or the spine portion may be formed during an integral moulding process.

The precise number of segments and living hinge elements of either the sheath portion or the spine portion is not particularly limited and may be selected based on e.g. the intended length of the bendable member. In some arrangements, each of the sheath portion and the spine portion may comprise more than 10 segments and respective living hinge elements, for example they may each comprise more than 15, more than 20, more than 25 or more than 30 segments and respective living hinge elements. In some arrangements, the number of segments and respective living hinge elements may be the same for the sheath portion as for the spine portion. In other arrangements, the number of segments and respective living hinge elements may differ between the sheath portion and the spine portion.

At least some of the spine living hinge elements and the sheath living hinge elements may be substantially aligned in one or both of the axial and/or the circumferential direction of the bendable member. That is, some or all of the series of spine living hinge elements and the series of sheath living hinge elements may be arranged at substantially the same positions along an axial length of the bendable member. Some or all of the series of spine living hinge elements and the series of sheath living hinge elements may be arranged at substantially the same positions about a circumferential direction of the bendable member. By providing an arrangement in which at least a portion of the spine living hinge elements and the sheath living hinge elements are substantially aligned in one or both of the axial and/or the circumferential direction of the bendable member, bending of the bendable member may be facilitated. Bending performance of the bendable member may be diminished if a large number of spine living hinge elements and the sheath living hinge elements are substantially misaligned.

Further optional features of the sheath portion will now be described.

The shape of the sheath portion is preferably selected to allow suitable bending function of the bendable member, whilst providing suitable protection and support for other components of an endoscope which may be incorporated in the bendable member when used in an endoscope.

The sheath ring segments may each comprise a radially outer surface, a radially inner surface, and first and second opposing sidewall surfaces. A first sidewall surface of a first sheath ring segment may be arranged to face a second sidewall surface of an adjacent second sheath ring segment. The sheath living hinge elements may be connected to the sheath ring segments at the first and second opposing sidewall surfaces of adjacent sheath ring segments.

In preferred arrangements, the living hinge elements of the sheath portion may comprise one or more flexible web portions, the term ‘web portion’ being used herein to refer to a thin strip of material. For example, each living hinge element may comprise two or more flexible web portions. Where there are two or more flexible web portions, these may be substantially equiangularly spaced about the bendable member. For example, where a living hinge element is constituted by a pair of flexible web portions, these may be disposed to be circumferentially about 180° apart about the sheath portion. For example, they may be oppositely disposed on an outer circumference of the sheath portion. Each selected living hinge element of the sheath portion may be circumferentially angularly offset with respect to an immediately adjacent living hinge element of the sheath portion. Each selected living hinge element of the sheath portion may be oriented at 90° with respect to an immediately adjacent living hinge element when viewed in cross-sections perpendicular to the longitudinal axial direction of the bendable member, i.e. may be angularly offset by 90° with respect to an immediately adjacent living hinge element. As will be readily understood, all references herein to components being oriented at X ° with respect to another component or feature when viewed in cross-sections perpendicular to the longitudinal axial direction of the bendable member shall be understood as defining an angular offset of X ° about the longitudinal axis of the relevant features.

In one preferred arrangement, each sheath living hinge element comprises a pair of flexible web portions extending from one sidewall surface and extending parallel to the axial direction of the sheath portion, wherein the flexible web portions are disposed to be circumferentially about 180° apart on the sidewall surface. The flexible web portions extend across a gap between axially adjacent sheath ring segments to attach to the axially adjacent sheath ring segment at a sidewall surface. The living hinge element (i.e. the pair of flexible web portions) of each selected sheath ring segment is circumferentially angularly offset with respect to the living hinge element (i.e. the pair of flexible web portions) of an immediately axially adjacent sheath ring segment by 90°, when viewed in a cross-section perpendicular to the axial direction of the bendable member. The effect of this is that the sheath ring segments of each pair of adjacent sheath ring segments can move relative to each other by bending of the pair of flexible web portions, allowing bending in one of two mutually perpendicular planes for each pair of adjacent sheath ring segments. This arrangement can provide for a suitable controllable yet flexible bending response of the sheath portion and the spine portion.

The size of the sheath ring segments is not particularly limited and may be selected as appropriate for the specific application intended. However, for preferred applications, the sheath portion may have an outer diameter of from 3 mm to 15 mm. For example, the sheath portion may have an outer diameter of 3 mm or more, 4 mm or more, 5 mm or more, 6 mm or more, 7 mm or more, 8 mm or more, 9 mm or more or 10 mm or more. The sheath portion may have an outer diameter of 15 mm or less, 13 mm or less, 12 mm or less, 11 or less or 10 mm or less. The sheath portion may have an inner diameter of from 1 mm to 13 mm. For example, the sheath portion may have an inner diameter of 1 mm or more, 2 mm or more, 3 mm or more, 4 mm or more, 5 mm or more, 6 mm or more, 7 mm or more, 8 mm or more, or 10 mm or more. The sheath portion may have an inner diameter of 13 mm or less, 12 mm or less, 11 mm or less, 10 or less or 9 mm or less.

In a particularly preferred arrangement, the sheath portion may have an inner diameter of about 6.5 mm, and an outer diameter of about 9.0 mm. This size may be particularly suitable for application of the bendable member as a component in a gastroscope, e.g. a transoral or transnasal gastroscope.

Each selected ring segment may be substantially identical to one or more other ring segments, such as to an adjacent ring segment, other than for its orientation. For example, each selected ring segment may be oriented at 90° with an adjacent ring segment when viewed in a cross-section perpendicular to the longitudinal axial direction of the bendable member.

In some arrangements, the sheath portion may comprise two terminal ring segments one on either end of the bendable member. A distal terminal ring segment may be provided at a distal end of the bendable member, said distal terminal ring segment being configured for connection to the distal tip assembly on an endoscope. A proximal terminal ring segment may be provided at a proximal end of the bendable member, said proximal terminal ring segment being configured for connection to an adjacent portion of the insertion section. One or more of the terminal ring segments may be different in size or shape to the remaining ring segments of the sheath portion (also referred to as ‘intermediate ring segments’).

One or more of the sheath ring segments may comprise a chamfered wall portion. The term ‘chamfered wall portion’ is used herein to refer to a transitional wall portion or edge between two surfaces. Said chamfered wall portion may be formed between the radially outer surface and a sidewall surface of the sheath ring segment. Said chamfered wall portion may be formed adjacent a living hinge element. In some arrangements, one or more of the sheath ring segments, or all of the sheath ring segments, may comprise a plurality of chamfered wall portions. Said chamfered wall portions may be equiangularly spaced about the circumference of the respective sheath ring segment. In some arrangements, one or more of the sheath ring segments, or all of the sheath ring segments, may comprise a two pairs of chamfered wall portions. A first pair of chamfered wall portions may be formed between the radially outer surface and the first sidewall surface of the sheath ring segment, and oppositely disposed about a circumference of the sheath ring segment. A second pair of chamfered wall portions may be formed between the radially outer surface and the second sidewall surface of the sheath ring segment, and oppositely disposed about a circumference of the sheath ring segment. The first and second pairs of chamfered wall portions may be oriented at 90° with respect to one another when viewed in a cross-section perpendicular to the longitudinal axial direction of the bendable member. Provision of such arrangements can allow for the sheath portion to achieve smaller bending radii than when one or more such chamfered wall portions are not provided, as impingement of adjacent sheath ring portions on one another during bending can be reduced or avoided.

The chamfered wall portion may comprise a planar chamfer (i.e. the chamfered wall portion may lie in a single plane). Alternatively, the chamfered wall portion may comprise a curved (non-planar) chamfer.

The chamfered wall portion may be formed at an angle of greater than 0° and less than 90°, between two faces of the sheath ring segment. In preferred arrangements, the chamfer may be formed at an angle of at 10° or more, 20° or more, 30° or more, or 40° or more between two faces of the sheath ring segment. The chamfered wall portion may be formed at an angle of 80° or less, 70° or less, 60° or less, or 50° or less between two faces of the sheath ring segment. In preferred arrangement, the chamfered wall portion may be formed at an angle of about 45° between two faces of the sheath ring segment. Where the chamfered wall portion is formed between the radially outer surface and a sidewall surface of the sheath ring segment, this angle may be measured between a plane tangential to the radially outer surface of the sheath ring segment, and a plane in which the chamfered wall portion lies.

The axial length of the chamfered wall portion may be 10% or more of the axial length of a single sheath ring segment. For example, the axial length of the chamfered wall portion may be 20% or more, 30% or more, or 40% or more of the axial length of a sheath ring segment. It may be preferred for the axial length of the chamfered wall portion to be no more than 50% of the axial length of the chamfered wall portion, as such arrangements may compromise the mechanical strength of the ring segment. For an arrangement in which the chamfered wall portion is formed between the radially outer surface and a sidewall surface of the sheath ring segment, the axial length of the chamfered wall portion may be measured as the length component of the chamfered wall portion measured in an axial direction in a plane tangential to the radially outer surface of the sheath ring segment.

Where present, each of the chamfered wall portions may extend angularly around the sheath ring segment for 10° or more, for example 20° or more, 30° or more, 40° or more, 50° or more, 60° or more, 70° or more, 80° or more, or 90° or more. In preferred arrangements, the angular extend of the chamfered wall portion is in a range of from 30° to 120°. The angular extent of the chamfered wall portion may be less than 180°, for example 170° or less, 160° or less, 150° or less, 140° or less, 130° or less, or 120° or less. In other words, the chamfered wall portion may not extend around the entire sheath ring segment: the sheath ring segment may comprise one or more substantially non-chamfered edge portions adjacent the chamfered wall portions.

In one preferred arrangement, each sheath ring segment comprises two pairs of oppositely disposed chamfered wall portions oriented at 90° with respect to one another when viewed in a cross-section perpendicular to the longitudinal axial direction of the bendable member, wherein each chamfered wall portion extends angularly for more than 90° around the circumference of the sheath ring segment, such that at least part of the first pair of chamfered wall portions and the second pair of chamfered wall portions overlap in a circumferential direction about the circumference of the sheath ring segment (albeit on opposite sides of the sheath ring segment). This arrangement has been found to be particularly advantageous as it can allow enhanced flexibility of the bending member during bending. Furthermore, where an outer casing is provided, the larger gaps between sheath ring segments provided by the larger chamfers can allow the outer casing to deflect into the chamfered areas without restricting the tight curl of the whole assembly.

It will be appreciated that provision of one or more such chamfered wall portions may have advantages even in arrangements not according to Innovation A or Innovation B as discussed herein.

Accordingly, a further independent aspect of the present disclosure is a bendable member for an imaging endoscope, said bendable member having a longitudinal axial direction, a circumferential direction and a radial direction, wherein the bendable member comprises:
a sheath portion comprising a series of sheath ring segments formed integrally with sheath living hinge elements, the sheath portion being bendable by bending of the sheath living hinge elements;
wherein some or all or said sheath ring segments comprise at least one chamfered wall portion.

The material composition of the sheath portion is not particularly limited and may be selected based on manufacturing considerations or the intended use and function of the bendable member. However, in preferred arrangements, the sheath portion is formed from one or more polymeric materials, for example a material selected from the group consisting of polyesters, polyamides (e.g. nylons), acetyls and elastomers. Nylon may be particularly preferred. Polymeric materials are generally cheap, are relatively facile to shape and/or mould during manufacture, and can offer good bending flexibility.

The method of manufacture of the sheath portion is not particularly limited and may be selected based on manufacturing considerations or the intended use and function of the bendable member. In some arrangements, the sheath portion is formed by injection moulding. In other arrangements, the sheath portion is formed by extrusion of a substantially cylindrical member, followed by local material removal to form the living hinge elements and/or shape the sheath ring segments.

Further optional features of the spine portion will now be described.

The spacer segments may each comprise a body having a central aperture. The central aperture may be substantially cylindrical. Each respective central aperture of the series of radially extending spacer segments may be substantially aligned to thereby define a spine portion central lumen. The central aperture of the spacer segment body may be configured to receive one or more components of an endoscope, for example it may be configured to receive one or more of e.g. a vacuum tube, a fluid conduit, or a sensor cable. The size of central aperture is not particularly limited and may be selected as appropriate for the specific application intended. In some cases the central aperture may have a diameter of from 0.5 mm to 5 mm, more preferably from 1 mm to 4 mm. For example, the central aperture may have a diameter of 1 mm or more, 2 mm or more, or 3 mm or more. The central aperture may have a diameter of 5 mm or less, or 4 mm or less. The central aperture may have a diameter of about 3.5 mm.

The spacer segments may comprise a radially outer surface, a radially inner surface, and first and second opposing sidewall surfaces. A first sidewall surface of a first spacer segment may be arranged to face a second sidewall surface of an adjacent second spacer segment. The spine living hinge elements may be connected to the spacer segments at the first and second opposing sidewall surfaces.

In preferred arrangements, the living hinge elements of the spine portion may comprise one or more flexible web portions, the term ‘web portion’ being used herein to refer to a thin strip of material. For example, each living hinge element may comprise two or more flexible web portions. Where there are two or more flexible web portions, these may be substantially equiangularly spaced about the bendable member. For example, where a living hinge element is constituted by a pair of flexible web portions, these may be disposed to be circumferentially about 180° apart about the spine portion. Each selected living hinge element of the spine portion may be circumferentially angularly offset with respect to an immediately adjacent living hinge element of the spine portion. Each selected living hinge element of the spine portion may be oriented at 90° with respect to an immediately adjacent living hinge element of the spine portion when viewed in a cross-section perpendicular to the longitudinal axial direction of the bendable member.

In one preferred arrangement, each spine living hinge element comprises a pair of flexible web portions extending from one sidewall surface and extending parallel to the axial direction of the spine portion, wherein the flexible web portions are disposed to be circumferentially about 180° apart on the sidewall surface. The flexible web portions extend across a gap between axially adjacent spacer segments to attach to the axially adjacent spacer segments at a sidewall surface. The living hinge element (i.e. the pair of flexible web portions) of each selected spacer segment is circumferentially angularly offset with respect to the living hinge element (i.e. the pair of flexible web portions) of an immediately axially adjacent spacer segment by 90°, when viewed in a cross-section perpendicular to the axial direction of the bendable member. The effect of this is that the spacer segments of each pair of adjacent spacer segments can move relative to each other by bending of the pair of flexible web portions, allowing bending in one of two mutually perpendicular planes for each pair of adjacent spacer segments. This arrangement can provide for a suitable controllable yet flexible bending response of the spine portion.

The size of the spacer segments is not particularly limited and may be selected as appropriate for the specific application intended. However, for preferred applications, the spacer segments may have an outer diameter as measured across the widest point of the segment of from 2 mm to 10 mm, more preferably about 4-8 mm, more preferably about 5-6 mm. For example, the spacer segments may have an outer diameter as measured across the widest point of the segment of 2 mm or more, 3 mm or more, 4 mm or more, 5 mm or more, or 6 mm or more. The sheath portion may have an outer diameter of 10 mm or less, 9 mm or less, 8 mm or less, 7 or less or 6 mm or less. This size may be particularly suitable for application of the bendable member as a component in a gastroscope, e.g. a transoral or transnasal gastroscope.

Each selected spacer segment may be substantially identical to one or more other spacer segments, or to an adjacent spacer segment, other than for its orientation. For example, each selected spacer segment may be oriented at 90° with an adjacent spacer segment when viewed in a cross-section perpendicular to the longitudinal axial direction of the bendable member.

In some arrangements, the spine portion may comprise two terminal spacer segments, one on either end of the bendable member: a distal terminal spacer segment disposed at a distal end of the bendable member and configured for connection to a distal tip assembly on an endoscope, and a proximal terminal spacer segment disposed at a proximal end of the bendable member and configured for connection to an adjacent portion of the insertion section. One or more of the terminal spacer segments may be different in size or shape to the remaining spacer segments of the spine portion (also referred to as ‘intermediate spacer segments’).

The one or more longitudinally extending conduits defined between the spacer segments of the spine portion and the sheath ring segments of the sheath portion may be defined between the radially outer surface of the spacer segment body and the radially inner surface of the sheath portion.

In preferred arrangements, the spacer segment body comprises one or more spacer arms which extend in a substantially radial direction and are configured to space at least a part of the radially outer surface of the spacer segment body and the radially inner surface of the sheath portion to thereby provide the one or more longitudinally extending conduits as a space adjacent said arm(s). The spacer segment body may comprise two or more spacer arms, three or more spacer arms, or four or more spacer arms. Where there are multiple spacer arms, the spacer arms may be equiangularly spaced about the circumference of the spacer segment body. Such conduits may be configured to receive one or more components of an endoscope, for example a sensor wire, or a fluid conduit. The circumferential distance between spacer arms may be from about 1 mm to about 5 mm, depending on the size of the spacer segment, and the number of spacer arms. In some arrangements, the circumferential distance between spacer arms may be about 2-3 mm.

Alternatively or additionally, the spacer segment body comprises one or more grooves or slots formed in the radially outer surface of the body, such that one or more longitudinally extending conduits are defined between the groove or slot of the spacer segment body and the radially inner surface of the sheath portion. Such conduits may be configured to receive one or more steering wires of an endoscope. In this way, such conduits can act as steering wire guides. These grooves may have a width of from about 0.5 mm to about 3 mm, more preferably from about 1 mm to about 2 mm.

In particularly preferred arrangements, the spacer segments comprise a plurality of spacer arms (for example four spacer arms), wherein at least one of said spacer arms comprises one or more slots formed at a radially outer extent of the arm. In some arrangements each arm may comprise one or more slots formed at a radially outer extent of the arm. In this way, it is possible to provide a plurality of longitudinally extending conduits between the spacer segments of the spine portion and the sheath ring segments of the sheath portion: a first set of conduits defined between each adjacent pair of spacer arms and the sheath portion, and a second set of conduits defined between the slot(s) formed at a radially outer extent of the arm(s) of the spacer segment body and the radially inner surface of the sheath portion.

The number of longitudinally extending conduits defined between the spacer segments of the spine portion and the sheath ring segments of the sheath portion is not particularly limited. There may be provided at least three longitudinally extending conduits extending along the bendable member between the spacer segments of the spine portion and the sheath ring segments of the sheath portion. There may be provided at least four, five, six, seven, eight or nine longitudinally extending conduits extending along the bendable member between the spacer segments of the spine portion and the sheath ring segments of the sheath portion. The of longitudinally extending conduits may be provided substantially equiangularly spaced about the bendable member when viewed in cross section. The longitudinally extending conduits may extend substantially along an entire length of the bendable member.

In some arrangements, some or all of the spacer segments may comprise one or more projections formed on at least one of the first or the second opposing sidewall surfaces. In a preferred arrangement, each spacer segment comprises at least one projection on both its respective first and second opposing sidewall surfaces. The one or more projections may project in a longitudinal direction from the first and/or second opposing sidewall surface. Where more than one projection is provided on a single sidewall surface, the projections may be substantially equiangularly spaced. Providing such projections on the sidewall surfaces of the spacer segments reduces the open spacing between adjacent spacer segments. This can help to reduce the risk of any components or tools located within the central aperture of the spacer segment from getting caught in the gaps between spacer segments, thus improving both safety and ease of use of e.g. an endoscope incorporating the bendable member. Whilst a similar effect of reducing the open spacing between adjacent spacer segments could be provided by reducing the length of the living hinge elements between adjacent segment, such a solution is not preferred, because changing the length of the living hinge elements may affect the bending response of the spine portion. By providing one or more projections as discussed above, it is therefore possible to reduce the risk of any components or tools located within the central aperture of the spacer segment from getting caught in the gaps between spacer segments, without substantially affecting the bending response of the spine portion.

The projections may take any suitable form, and the shape of the projections is not particularly limited. For example, the projections may be cylindrical, cuboid, approximately cuboid (for example, curved cuboid).

The projections may extend, for example, for 0.3 mm or more, 0.4 mm or more, 0.5 mm or more, 0.6 mm or more, 0,7 mm or more, 0.8 mm or more, 0.9 mm or more, 1 mm or more, 2 mm, 3 mm or more, 4 mm or more, or 5 mm or more into the open spacing between adjacent spacer segments. The extent of the projection in absolute terms may be selected based on the size of the bendable member (and accordingly the size of the opening between adjacent spacer segments).

The projection(s) may be arranged to occlude at 1% or more, 5% or more, 10% or more, 15% or more, 20% or more, 30% or more, or more of an area of an opening between adjacent spacer segments. They may be arranged to occlude no more than about 50%, or no more than about 40% of said area, to avoid the projection(s) impinging on one another, or on other parts of the spacer segment during bending of the bendable member.

It will be appreciated that provision of one or more such projections formed on at least one sidewall surface of the spacer segments may have advantages even in arrangements not according to Innovation A or Innovation B as discussed herein. Accordingly, a further independent aspect of the present disclosure is a bendable member for an imaging endoscope, said bendable member having a longitudinal axial direction, a circumferential direction and a radial direction, wherein the bendable member comprises:
a series of constructional elements arranged longitudinally in series along the bendable member and formed integrally with a plurality of living hinge elements that permit bending between adjacent constructional elements, each constructional element comprising at least a radially outer surface and first and second opposing sidewall surfaces,
wherein some or all of the constructional elements comprise one or more projections formed on at least one of the first or the second opposing sidewall surfaces.

The material composition of the spine portion is not particularly limited and may be selected based on manufacturing considerations or the intended use and function of the bendable member. However, in preferred arrangements, the spine portion is formed from one or more polymeric materials, for example a material selected from the group consisting of polyesters, polyamides (e.g. nylons), acetyls and elastomers. Nylon may be particularly preferred. Polymeric materials are generally cheap, are relatively facile to shape and/or mould during manufacture, and can offer good bending flexibility.

The method of manufacture of the spine portion is not particularly limited and may be selected based on manufacturing considerations or the intended use and function of the bendable member. In some arrangements, the spine portion is formed by injection moulding. In other arrangements, the spine portion is formed by extrusion of a substantially cylindrical member, followed by local material removal to form the living hinge elements and/or shape the spine portion spacer segments.

The spine portion and the sheath portion may be formed from the same material, or they may be formed from different materials. In some arrangements, the materials of the spine portion and the sheath portion may be selected as materials with different bending modulus (for example as measured according to a flexural test such as the ASTM D790). By selecting to use different materials for the spine portion and the sheath portion, the bending response of the bendable member can be suitably adjusted. For example, the spine portion may be formed from a comparatively more rigid material than the sheath portion. This can allow for the bending response of the bendable member to be mainly controlled by the bending response of the spine portion.

The sheath portion and the spine portion may be configured to interact to reduce or prevent relative rotational movement of the sheath portion and spine portion. In particular it may be desirable to prevent such moment when the sheath portion and the spine portion are assembled as part of a bendable member forming part of an endoscope. For example, the sheath portion and the spine portion may have one or more cooperating parts which engage with one another to prevent such relative movement. In preferred arrangements, a terminal constructional element (i.e. ring segment or spacer segment) of the sheath portion or the spine portion comprises a flange portion configured to interlock with a corresponding recess formed on a terminal constructional element of the other of the sheath portion or spine portion.

The bendable member may comprise an outer casing. The outer casing may comprise a tube arranged to surround the sheath portion of the bendable member. Provision of an outer casing or sheath can reduce risk of inadvertent damage to the articulated member during use. Preferably the outer casing is formed from a flexible and/or elastomeric material. This can allow for relatively unimpeded bending of the articulated member, as the outer casing can stretch and/or fold as appropriate during bending. Some preferred materials include silicones and/or thermoplastic elastomers (TPE).

As discussed above, the bendable member is for an imaging endoscope. In particular, bendable members according to the present invention may find application as a component of an insertion section of the endoscope, and/or as a component of a steering section of the endoscope.

Accordingly, in a second aspect of Innovation A, the present invention provides an imaging endoscope, or an imaging endoscopy system, comprising:
a hand controller;
an insertion section having a proximal end connected to the hand controller and a distal end for insertion into a subject, wherein a distal tip assembly is located at the distal end of the insertion section and a steering section is located adjacent and proximal to the distal tip, said steering section being bendable for steering by operation of the hand controller;
wherein at least a part of the insertion section comprises a bendable member having an longitudinal axial direction, a circumferential direction and a radial direction, wherein the bendable member comprises:
a sheath portion comprising a series of sheath ring segments formed integrally with sheath living hinge elements, the sheath portion being bendable by bending of the sheath living hinge elements; and
a spine portion comprising a series of radially extending spacer segments formed integrally with spine living hinge elements, the spine portion being bendable by bending of the spine living hinge elements; wherein
the spine portion and the sheath portion are coaxially arranged such that one or more longitudinally extending conduits are defined between the spacer segments of the spine portion and the sheath ring segments of the sheath portion.

The part of the insertion section comprising the bendable member may include the steering section, Alternatively or additionally, the part of the insertion section comprising the bendable member may include a part of the insertion section intermediate the proximal end of the insertion section and the steering section (this part also referred to herein as a ‘insertion section main body’).

The insertion section may include a single bendable member, or it may include two or more bendable members. For example, in one arrangement, the insertion section may comprise a single bendable member or two or more bendable members constituting a part of the steering section. In another arrangement, the insertion section may comprise a single bendable member or two or more bendable members constituting a part of the insertion section main body. In another arrangement, the insertion section may comprise a bendable member which extends along substantially the entire length of the insertion section, between the proximal end of the insertion section and the distal tip assembly. In another arrangement, the insertion section may comprise a first bendable member constituting part of the steering section, and a second bendable member constituting a part of the insertion section main body. Typically in a conventional reusable endoscope, the part of the insertion section intermediate the proximal end and the steering section (the insertion section main body) would include an outer part formed of one or two helically wound flat springs which are essentially tubular, which are over-braided with a braided fibre layer and then covered by an outer smooth sleeve extruded over the braid. Arrangements according to the present invention in which this helical spring arrangement is replaced with a bendable member as described above can provide reduced manufacturing complexity in comparison to these known arrangements.

The hand controller may be configured to be releasably connectable to a base unit by means of an umbilical section. The base unit may provide electrical power, irrigation liquid and insufflation gas to the insertion section, via the umbilical section and hand controller.

The distal tip assembly may include a light source for illumination of a region of tissue of interest. Preferably, the light source includes at least one light emitting diode (LED). There may be provided at least one electrical conductor along the insertion section for providing electrical power to the light source.

The distal tip assembly may further include an imaging chip for imaging the region of tissue of interest. There may be provided at least one electrical conductor along the insertion section for providing electrical power to the imaging chip. There may be provided at least one electrical conductor for conducting electrical signals from the imaging chip to the proximal end of the insertion section. In this manner, it is possible to implement an imaging endoscope system in which there is no need for the insertion section to transmit light, whether as illumination or optical images.

The distal tip may include a distal tip housing comprising light-transmissive portion. The distal tip housing may be integrally formed from a polymeric material.

The distal tip housing may have a collar portion extending proximally of a distal end face. The collar portion may be adapted to fit over a distal end of the steering section. This is considered to be a convenient implementation.

The distal tip housing may have a cleaning nozzle arranged at the distal end face, to direct irrigation liquid to clean a lens of the imaging chip.

The insertion section may comprise at least one steering wire, fixed at or near the distal end of the insertion section, and extending along the length of the insertion section for connection to the hand controller, whereby the steering section of the insertion section is bendable for steering by application of tension to the at least one steering wire by operation of the hand controller. There may be provided such four steering wires, the four steering wires being substantially equiangularly spaced around the insertion section when viewed in cross section.

The steering wires may have sheaths each defining an axis of constrained movement for each respective steering wire. In other words, preferably the steering wires are provided as Bowden cables. Preferably, the steering wires extend through the steering section to the distal tip and the sheaths of the steering wires do not extend through the steering section. In this way, application of tension to the one or more steering wires can cause bending primarily in the steering section of the insertion section, without substantially influencing the curvature of the remaining portion of the insertion section intermediate the hand controller and the steering section.

Preferably the steering wire(s) are at least partly located in the one or more longitudinally extending conduits defined between the spacer segments of the spine portion and the sheath ring segments of the sheath portion of the bendable member. In this way, unwanted movement of the steering wires within the insertion section can be reduced or prevent, with the longitudinally extended conduits acting as steering wire guides.

As discussed above, the number of longitudinally extending conduits defined between the spacer segments of the spine portion and the sheath ring segments of the sheath portion of the bendable member is not particularly limited. In preferred arrangements, at least one longitudinally extending conduit may be designated for use to conduct a flow of irrigation liquid to the distal tip, for example by being configured to receive an irrigation liquid tube. At least one longitudinally extending conduit may be designated for use to conduct a flow of insufflation gas to the distal tip, for example by being configured to receive an insufflation gas tube. At least one longitudinally extending conduit may be designated for use to provide suction at the distal tip. At least one longitudinally extending conduit may be designated for use to conduct a surgical instrument to the distal tip. Alternatively or additionally, one or more of these endoscope functions may be provided through a central lumen of the spine portion of the bendable member.

The imaging endoscopy system 100 may be provided in kit form, including a single base unit 2, and a plurality of sealed containers which together contain a plurality of hand controllers 6, in sterile condition, a plurality of umbilical sections 4, in sterile condition, and a plurality of insertion sections 10, in sterile condition. The hand controller 6 and the umbilical section 4 and the insertion section 10 may be formed integrally in the sense that they are assembled together in the factory.

When provided as a kit, for assembly of the imaging endoscopy system and readying of this for use in an endoscopic procedure, the base unit 2 is located in a suitable location for an endoscopic procedure, an operator (an endoscopist, or an assistant) opens at least one of the sealed containers, and extracts the hand controller 6, in sterile condition, the umbilical section 4, in sterile condition, and the insertion section 10, in sterile condition. The sterile umbilical section 4 is then connected to the base unit 2 via connector 8 to provide an imaging endoscopy system 100 ready for use.

Other features of the imaging endoscope, or imaging endoscopy system may be implemented as described in GB2569013 B—Imaging endoscope system and associated methods, published 9 Sep. 2020, which is herein incorporated by reference.

In a third aspect of Innovation A, the present invention provides a method of assembly of an imaging endoscope including steps of:
providing a spine portion of a bendable member, the spine portion comprising a series of radially extending spacer segments formed integrally with spine living hinge elements, the spine portion being bendable by bending of the spine living hinge elements;
arranging one or more endoscope components on said spine portion;
providing a sheath portion of a bendable member comprising a series of sheath ring segments formed integrally with sheath living hinge elements, the sheath portion being bendable by bending of the sheath living hinge elements; and
coaxially arranging the spine portion and sheath portion of the bendable member such that one or more longitudinally extending conduits are defined between the spacer segments of the spine portion and the sheath ring segments of the sheath portion, and such that the one or more endoscope components are at least partly located in said longitudinally extending conduits.

The above steps may be performed in the order stated above. That is, in some embodiments, the step of arranging the one or more endoscope components on said spine portion is performed before the step of coaxially arranging the spine portion and sheath portion of the bendable member.

The one or more endoscope components may be selected from one or more of a steering wire, an electrical wire or sensor cable, a fluid conduit, a vacuum tube, or any other endoscope component configured to extend along the length of the insertion section of the tube.

Methods of assembly of an endoscope according to the present invention may be faster than conventional methods of assembly of conventional endoscopes, in which one or more components of the endoscope are required to be threaded along the length of an insertion/steering section of the endoscope.

### Innovation B

In a first aspect of Innovation B, the present invention provides a bendable member for an imaging endoscope, said bendable member comprising a series of constructional elements arranged longitudinally in series along the bendable member and formed integrally with a plurality of living hinge elements that permit bending between adjacent constructional elements, wherein, when the bendable member is straight, a first living hinge element of the plurality of living hinge elements has a different bending stiffness to a second living hinge element of the plurality of living hinge elements.

The term ‘living hinge element’ is used herein to define a flexible hinge integrally formed with, and preferably made from the same material as, the two constructional elements it connects. The living hinge elements may be formed by local material removal (e.g. cutting) of the bendable member. Alternatively, the living hinge elements may be formed during an integral moulding process.

The form of the constructional elements is not particularly limited and may be selected based on the intended application of the bendable member. The constructional elements may comprise a body having at least a radially outer surface, and first and second opposing sidewall surfaces. A first sidewall surface of a first constructional element may be arranged to face a second sidewall surface of an adjacent constructional element. The living hinge elements may be connected to the constructional elements at the first and second opposing sidewall surfaces. In some arrangements, the constructional element may comprise a central aperture. Where they comprise a central aperture, the central aperture may define a radially inner surface of the constructional element. The constructional elements may comprise ring segments. For example, the bendable member may be a sheath portion as discussed above in relation to Innovation A. The constructional elements may comprise segments having one or more arms which extend in a substantially radial direction. For example, the bendable member may be a spine portion as discussed above in relation to Innovation A.

Each constructional element of the series of constructional elements may be substantially identical. Alternatively, the size or shape of the constructional elements may vary within the series of constructional elements. That is, a first constructional element of the series of constructional elements may have a different size or shape to a second constructional element of the series of constructional elements.

The bending stiffness of the living hinge elements may also be referred to as flexural rigidity, which is defined in the art as the force couple required to bend a fixed non-rigid structure by one unit of curvature. Where a first living hinge element has a greater bending stiffness than a second living hinge element, the force required to bend the first living hinge element to a predetermined curvature will be greater than the force required to bend the second living hinge element to the same predetermined curvature. Accordingly, for a given applied bending moment, the first living hinge element will exhibit less deflection than the second living hinge element. When assembled as part of an endoscope, bending forces on the steering section are typically applied by application of tension to one or more steering wires attached at a distal end of the steering section. The bending moment applied to each living hinge element may therefore vary along the length of the steering section. By providing an arrangement in which a first living hinge element of the plurality of living hinge elements has a different bending stiffness to a second living hinge element of the plurality of living hinge elements, the bending response of the living hinge elements bendable member can be tailored along the length of the bendable member. This may allow for the bendable member to be bent with e.g. a more uniform radius of curvature, by suitable selection of the bending stiffness of two or more living hinge elements along the length of the bonding control device of the steering section.

In some arrangements, the bending stiffness of some or all of the living hinge elements is selected to provide a predetermined mechanical response profile along the length of the bendable member. The mechanical response profile may include e.g. the bending resistance of the bendable member along the length of the bendable member, or the torsional stiffness of the bendable member along the length of the bendable member.

In some arrangements, a first set of living hinge elements may have a first predetermined bending stiffness, and a second set of living hinge elements may have a second predetermined bending stiffness, wherein the first and second predetermined bending stiffnesses are different. In some arrangements, there may be three or more sets of living hinge elements, four or more sets of living hinge elements, or five or more sets of living hinge elements, wherein each set of living hinge elements has a different bending stiffness to the bending stiffness of living hinge elements in the other sets of living hinge elements.

In other arrangements, the bending stiffness of each of the living hinge elements along the length of the bendable member may differ. For example the bending stiffness of each living hinge element along the length of the bendable member may be selected to provide a continuous spectrum of bending stiffnesses along said length: for example, an arrangement in which the bending stiffness continuously increases along the length, or continuously decreases along the length.

The precise form of the living hinge elements is not particularly limited, however in preferred arrangements, the living hinge elements may comprise one or more flexible web portions, the term ‘web portion’ being used herein to refer to a thin strip of material. For example, each living hinge element may comprise two or more flexible web portions. Where there are two or more flexible web portions, these may be substantially equiangularly spaced about the bendable member. For example, where a living hinge element is constituted by a pair of flexible web portions, these may be disposed to be circumferentially about 180° apart about the bendable member. Each selected living hinge element may be circumferentially angularly offset with respect to an immediately axially adjacent living hinge element of the bendable member. Each selected living hinge element may be oriented at 90° with respect to an immediately adjacent living hinge element when viewed in a cross-section perpendicular to the longitudinal axial direction of the bendable member.

In one preferred arrangement, each living hinge element comprises a pair of flexible web portions extending from one sidewall surface of a first constructional element and extending parallel to the axial direction of the bendable member, wherein the flexible web portions are disposed to be circumferentially about 180° apart on the sidewall surface of the constructional element. The flexible web portions extend across a gap between axially adjacent constructional elements to attach to the axially adjacent constructional element at a sidewall surface. The living hinge element (i.e. the pair of flexible web portions) of each selected constructional element is circumferentially angularly offset with respect to the living hinge element (i.e. the pair of flexible web portions) of an immediately axially adjacent constructional element by 90°, when viewed in a cross-section perpendicular to the axial direction of the bendable member. The effect of this is that the constructional elements of each pair of adjacent constructional elements can move relative to each other by bending of the pair of flexible web portions, allowing bending in one of two mutually perpendicular planes for each pair of adjacent constructional elements. This arrangement can provide for a suitable controllable yet flexible bending response of the bendable member.

The relative bending stiffness of the living hinge elements may be varied by varying at least one dimension of the living hinge elements. In other words, the first living hinge element may have at least one dimension which is different to a corresponding dimension of the second living hinge element. The length, width and/or thickness of the first living hinge element may be different to a corresponding length, width and/or thickness of the second living hinge element.

Where the living hinge elements comprise a pair of flexible web portions, each web portion having a length measured in an axial direction of the bendable member, a width measured in a circumferential direction of the bendable member, and a thickness measured in a radial direction of the bendable member, one or more of the following (i), (ii), or (iii) may apply:
(i) the length of the flexible webs of the first living hinge element may be different to the length of the flexible webs of the second living hinge element.
(ii) the width of the flexible webs of the first living hinge element may be different to the width of the flexible webs of the second living hinge element.
(iii) the thickness of the flexible webs of the first living hinge element may be different to the width of the flexible webs of the second living hinge element.

The pitch between adjacent living hinge elements may be substantially uniform along the length of the bendable member. The pitch may be measured as the distance between centres of adjacent living hinge elements. The pitch between adjacent living hinge elements may be from 1 mm to 20 mm, more preferably from 2 mm to 10 mm. For example it may be about 2 mm, 3 mm, 4 mm, 5 mm, 6 mm, 7 mm, 8 mm 9 mm, 10 mm, 11 mm, 12 mm, 13 mm, 14 mm, 15 mm, 16 mm, 17 mm, 18 mm, 19 mm or 20 mm. A pitch of about 5 mm may be particularly preferred.

In some arrangements, the bendable member consists of a single, integrally moulded part. In other arrangements, the bendable member may comprise first and second coaxially arranged parts, each of the coaxially arranged parts of the bendable member being formed from a series of constructional elements arranged longitudinally in series along the bendable member and formed integrally with a plurality of living hinge elements that permit bending between adjacent constructional elements, and wherein, for at least one of the coaxially arranged parts, or for both of the coaxially arranged parts, when the bendable member is straight, a first living hinge element of the plurality of living hinge elements of said part has a different bending stiffness to a second living hinge element of the plurality of living hinge elements of said part.

In preferred arrangements, the bendable member comprises first and second coaxially arranged parts, wherein:
the first part comprises a sheath portion comprising a series of sheath ring segments formed integrally with sheath living hinge elements, the sheath portion being bendable by bending of the sheath living hinge elements; and
the second part comprises a spine portion comprising a series of radially extending spacer segments formed integrally with spine living hinge elements, the spine portion being bendable by bending of the spine living hinge elements; wherein one or both of (i) and (ii) applies:
(i) a first sheath living hinge element has a different bending stiffness to a second sheath living hinge element;
(ii) a first spine living hinge element has a different bending stiffness to a second spine living hinge element.

Preferably the spine portion and the sheath portion are coaxially arranged such that one or more longitudinally extending conduits are defined between the spacer segments of the spine portion and the sheath ring segments of the sheath portion.

Optional features of this preferred arrangement are discussed above in relation to Innovation A, said features being equally applicable to Innovation B—in particular features relating to the shape, size and configuration of the sheath portion and the spine portion, and the arrangement of one or more longitudinally extending conduits defined between the spacer segments of the spine portion and the sheath ring segments of the sheath portion.

The bendable member may comprise an outer casing. The outer casing may comprise a tube arranged to surround the sheath portion of the bendable member. Provision of an outer casing or sheath can reduce risk of inadvertent damage to the articulated member during use. Preferably the outer casing is formed from a flexible and/or elastomeric material. This can allow for relatively unimpeded bending of the articulated member, as the outer casing can stretch and/or fold as appropriate during bending. Some preferred materials include silicones and/or thermoplastic elastomers (TPE).

The bendable member may alternatively be referred to herein as a ‘bending control device’, and vice versa. Preferably, the bendable member/bending control device forms a part of a steering section of an endoscope, in particular, an imaging endoscope.

Accordingly, in a second aspect of Innovation B, the present invention provides an imaging endoscope, or an imaging endoscopy system, comprising:
a hand controller;
an insertion section having a proximal end connected to the hand controller and a distal end for insertion into a subject, wherein a distal tip assembly is located at the distal end of the insertion section and a steering section is located adjacent and proximal to the distal tip, said steering section being bendable for steering by operation of the hand controller;
wherein at least a part of the insertion section comprises a bendable member comprising a series of constructional elements arranged longitudinally in series along the steering section and formed integrally with a plurality of living hinge elements that permit bending between adjacent constructional elements, wherein, when the steering section is straight, a first living hinge element of the plurality of living hinge elements has a different bending stiffness to a second living hinge element of the plurality of living hinge elements.

The part of the insertion section comprising the bendable member may include the steering section, Alternatively or additionally, the part of the insertion section comprising the bendable member may include a part of the insertion section intermediate the proximal end of the insertion section and the steering section (this part also referred to herein as a ‘insertion section main body’).

The insertion section may include a single bendable member, or it may include two or more bendable members. For example, in one arrangement, the insertion section may comprise a single bendable member or two or more bendable members constituting a part of the steering section, said bendable members being arranged longitudinally in series, or concentrically. In another arrangement, the insertion section may comprise a single bendable member or two or more bendable members constituting a part of the insertion section main body, said bendable members being arranged longitudinally in series, or concentrically. In another arrangement, the insertion section may comprise a bendable member which extends along substantially the entire length of the insertion section, between the proximal end of the insertion section and the distal tip assembly. In another arrangement, the insertion section may comprise a first bendable member constituting part of the steering section, and a second bendable member constituting a part of the insertion section main body. Typically in a conventional reusable endoscope, the part of the insertion section intermediate the proximal end and the steering section (the insertion section main body) would include an outer part formed of one or two helically wound flat springs which are essentially tubular, which are over-braided with a braided fibre layer and then covered by an outer smooth sleeve extruded over the braid. Arrangements according to the present invention in which this helical spring arrangement is replaced with a bendable member as described above can provide reduced manufacturing complexity in comparison to these known arrangements.

Where the insertion section main body comprises a bendable member, it may be preferred for the end of the insertion section main body proximal the hand controller to have a higher bending stiffness (i.e. a higher resistance to bending) than a portion of the insertion section main body distal to the hand controller. This can help to reduce damage to the insertion section main body during manipulation of the imaging endoscope during an endoscopy procedure by a user of the device. Such an arrangement may also increase the torsional stiffness of the insertion section main body, thereby allowing an operator to more easily transmit torque from the hand controller to the insertion section main body.

The hand controller may be configured to be releasably connectable to a base unit by means of an umbilical section. The base unit may provide electrical power, irrigation liquid and insufflation gas to the insertion section, via the umbilical section and hand controller.

The distal tip assembly may include a light source for illumination of a region of tissue of interest. Preferably, the light source includes at least one light emitting diode (LED). There may be provided at least one electrical conductor along the insertion section for providing electrical power to the light source.

The distal tip assembly may further include an imaging chip for imaging the region of tissue of interest. There may be provided at least one electrical conductor along the insertion section for providing electrical power to the imaging chip. There may be provided at least one electrical conductor for conducting electrical signals from the imaging chip to the proximal end of the insertion section. In this manner, it is possible to implement an imaging endoscope system in which there is no need for the insertion section to transmit light, whether as illumination or optical images.

The distal tip may include a distal tip housing comprising light-transmissive portion. The distal tip housing may be integrally formed from a polymeric material.

The distal tip housing may have a collar portion extending proximally of a distal end face. The collar portion may be adapted to fit over a distal end of the steering section. This is considered to be a convenient implementation.

The distal tip housing may have a cleaning nozzle arranged at the distal end face, to direct irrigation liquid to clean a lens of the imaging chip.

The insertion section may comprise at least one steering wire, fixed at or near the distal end of the insertion section, and extending along the length of the insertion section for connection to the hand controller, whereby the steering section of the insertion section is bendable for steering by application of tension to the at least one steering wire by operation of the hand controller. There may be provided such four steering wires, the four steering wires being substantially equiangularly spaced around the insertion section when viewed in cross section.

The steering wires may have sheaths each defining an axis of constrained movement for each respective steering wire. In other words, preferably the steering wires are provided as Bowden cables. Preferably, the steering wires extend through the steering section to the distal tip and the sheaths of the steering wires do not extend through the steering section. In this way, application of tension to the one or more steering wires can cause bending primarily in the steering section of the insertion section, without substantially influencing the curvature of the remaining portion of the insertion section intermediate the hand controller and the steering section.

Preferably the steering wire(s) are at least partly located in one or more longitudinally extending conduits defined within the bendable member—for example, in arrangement where the bendable member comprises first and second coaxially arranged parts, the steering wire(s) may be located in conduits defined between said parts. In this way, unwanted movement of the steering wires within the insertion section can be reduced or prevent, with the longitudinally extended conduits acting as steering wire guides.

The invention includes the combination of the developments, aspects of developments, and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1 shows a schematic view of an imaging endoscopy system according to an embodiment of the present invention.

FIG. 2 shows a perspective line drawing of part of an endoscope insertion section incorporating a bendable member according to an embodiment of the present invention.

FIG. 3 shows the same part of an endoscope insertion section as FIG. 2, with an outer casing component removed.

FIG. 4 shows a perspective line drawing of a sheath portion of a bendable member according to an embodiment of the present invention.

FIG. 5 shows a close-up view of a part of the same sheath portion as FIG. 4.

FIG. 6 shows a perspective line drawing of a spine portion of a bendable member according to an embodiment of the present invention.

FIG. 7 shows a close-up view of a part of the same spine portion as FIG. 6.

FIG. 8 shows a view from a cross-section of the same spine portion as FIG. 6, the cross section being taken perpendicular to the axial direction of the spine portion.

FIG. 9 shows a view from a cross-section of the same part of an endoscope insertion section as FIG. 2, the cross section being taken perpendicular to the axial direction of the insertion section.

FIG. 10 shows a perspective line drawing of part of an endoscope insertion section incorporating a bendable member according to a further embodiment of the present invention, with an outer casing component removed.

FIG. 11 shows a close-up view of a part of the sheath portion of the further embodiment shown in FIG. 10

FIG. 12 shows a perspective line drawing of a spine portion of a bendable member of the further embodiment shown in FIG. 10

FIG. 13 shows a close-up view of a part of the same spine portion as FIG. 12.

### DETAILED DESCRIPTION OF THE INVENTION

As set out above, the present inventors have realized a number of innovations which can reduce the overall cost and complexity of manufacture of an insertion section of an imaging endoscopy system, whilst providing suitable or improved performance in comparison to conventional endoscopes.

Aspects and embodiments of the present invention will now be discussed with reference to the accompanying figures. Further aspects and embodiments will be apparent to those skilled in the art. All documents mentioned in this text are incorporated herein by reference.

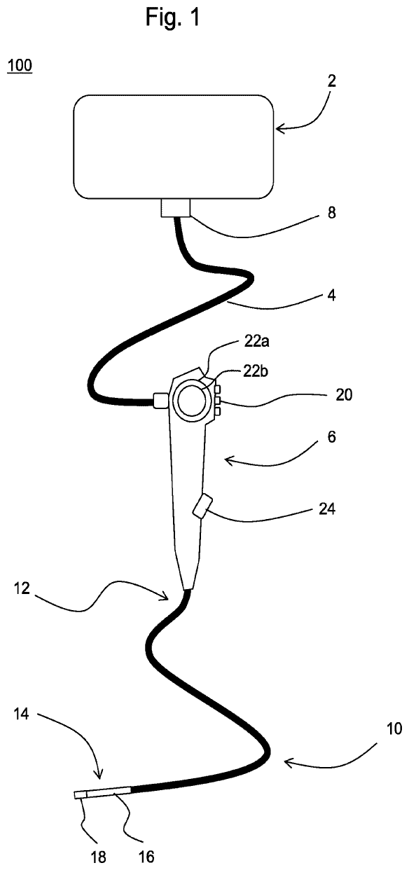

FIG. 1 shows a schematic view of an imaging endoscopy system 100 according to an embodiment of the present invention. The system includes a base unit 2 that is intended to be re-usable. Umbilical section 4 releasably connects the base unit 2 and hand controller 6 via a connector 8. Insertion section 10 has a proximal end 12 connected to the hand controller 6 and a distal end 14 for insertion into a subject. The distal end 14 has a steering section 16 and a distal tip assembly 18, which will be described in greater detail later.

The base unit 2 is operable to provide electrical power, irrigation liquid, insufflation gas and suction via the umbilical section 4 and hand controller 6 to the insertion section 10 in a manner well known in the in art. The delivery of insufflation gas, irrigation liquid and suction are controlled using mechanical valves 20 in the hand controller 6. These valves are referred to as “trumpet” type valves in view of their similarity in appearance and operation to valve keys on a trumpet. The insufflation gas, irrigation liquid and suction are supplied from a base unit at respective suitable fixed pressures. The timing of delivery and the rate of delivery to the distal end of the endoscope is then controlled by the user operating the mechanical valves at the hand controller. The valves operate in an analogue manner (allowing different flow rates of insufflation gas and irrigation liquid, for example, based on how far they are pressed by the user).

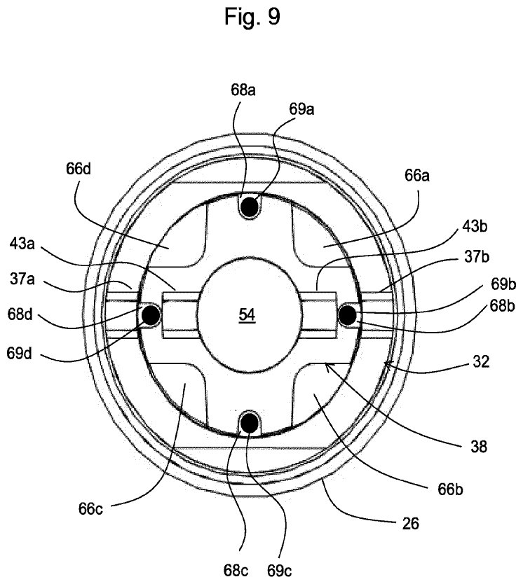

The hand controller 6 includes a steering control mechanism including first and second steering wire controls 22a, 22b, for controlling bending of the steering section 16. The first and second steering wire controls 22a, 22b are, in this arrangement, provided as first and second rotatable control wheels. The first and second steering wire controls 22a, 22b are operable by a user to apply tension to four steering wires (not shown in FIG. 1, but see e.g. FIG. 7 and FIG. 9 which show example arrangements of steering wires 69a, b, c, d) in order to control the bending of the steering section 16 in a known manner: rotation of the first rotatable control wheel winds a first pair of steering wires on or off a rotational drive wheel to thereby bend the steering section in a first plane, and rotation of the second rotatable control wheel winds a second pair of steering wires on or off a rotational drive wheel to thereby bend the steering section in a second plane, perpendicular to the first plane.

The steering wires 69a, b, c, d are provided as part of a Bowden cable, i.e. each wire is surrounded by a sheath defining an axis of constrained movement for each respective steering wire. The steering wires extend along the length of the insertion section 10, through the steering section 16 to the distal tip assembly 18. The sheaths of the steering wires do not extend through the steering section 16. In this way, application of tension to the steering wires can cause bending primarily in the steering section of the insertion section, without substantially influencing the curvature of the remaining portion of the insertion section intermediate the hand controller and the steering section.

The hand controller further comprises a tool insertion port 24 adapted to receive one or more surgical instruments, such as biopsy forceps.

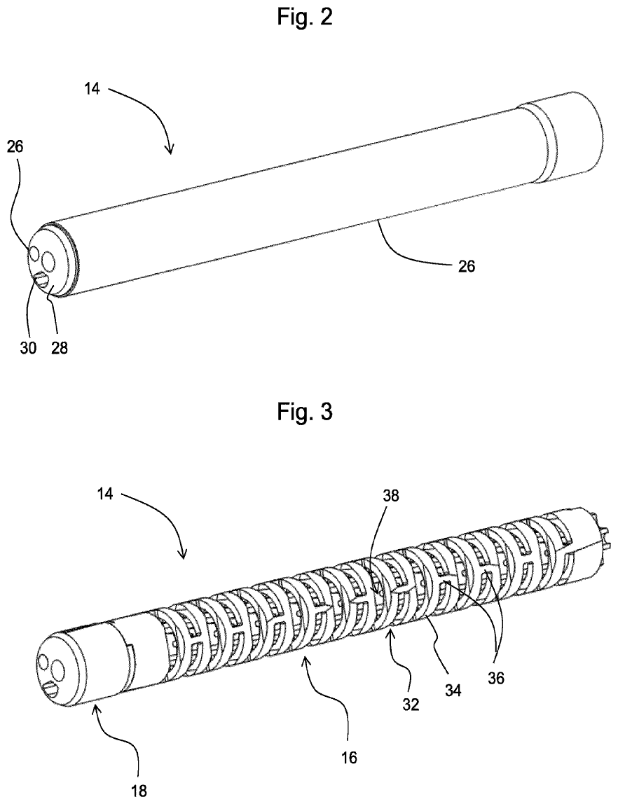

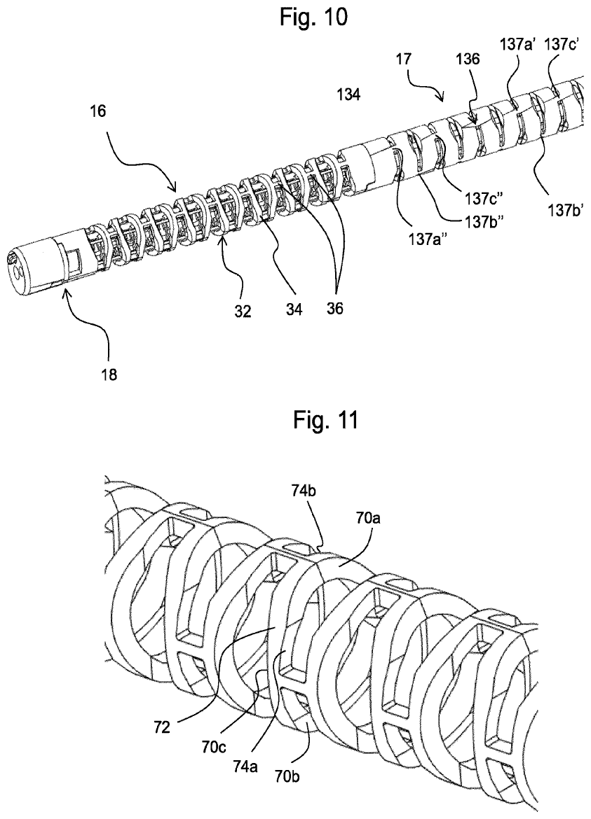

Features relating to the insertion section, and particularly to the distal end 14 of the insertion section will now be described in greater detail with reference to FIG. 2-13. FIGS. 2-9 relate to a first embodiment of the present disclosure. FIGS. 10-13 relate to a second embodiment of the present disclosure.

FIG. 2 shows a perspective line drawing of the distal end of the insertion section incorporating a bendable member according to the present invention. FIG. 3 shows the same part of an endoscope insertion section with an outer casing component removed. The distal end of the insertion section includes the steering section 16 and the distal tip assembly 18. An outer casing component 26 is provided to encase the distal end of the insertion section.

The distal tip assembly 18 includes a light source (not shown) provided within a lighting and imaging housing portion 26 on the distal end face 28 of the distal tip assembly, for illumination of a region of tissue of interest. Typically this light source is an LED. At least one electrical conductor (not shown) is provided along the insertion section 10 for providing electrical power to the light source. The distal tip assembly 18 further includes an imaging chip (not shown) provided within a lighting and imaging housing portion 26 for imaging the region of tissue of interest. The imaging chip is provided in the form of a camera, with an objective lens located to collect and direct light onto the imaging chip. An electrical conductor (not shown) is provided for conducting electrical signals from the imaging chip to the proximal end 12 of the insertion section 10.

The distal tip assembly 18 has a cleaning nozzle 30 arranged at the distal end face 28, to direct irrigation liquid to clean the lighting and imaging housing portion 26 during use.

As can be seen in FIG. 3, the steering section 16 includes a bendable member comprising first and second coaxially arranged parts, each of the coaxially arranged parts being formed from a series of constructional elements arranged longitudinally in series along the bendable member and formed integrally with a plurality of living hinge elements that permit bending between adjacent constructional elements.

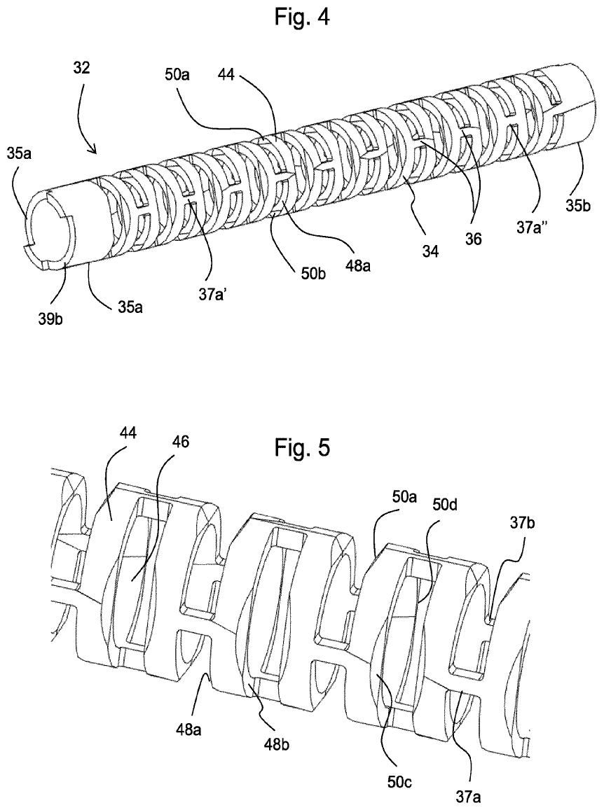

The first part comprises a sheath portion 32 comprising a series of constructional elements which are sheath ring segments 34 formed integrally with sheath living hinge elements 36, the sheath portion being bendable by bending of the sheath living hinge elements. The sheath portion is discussed in greater detail in relation to FIG. 4 and FIG. 5.

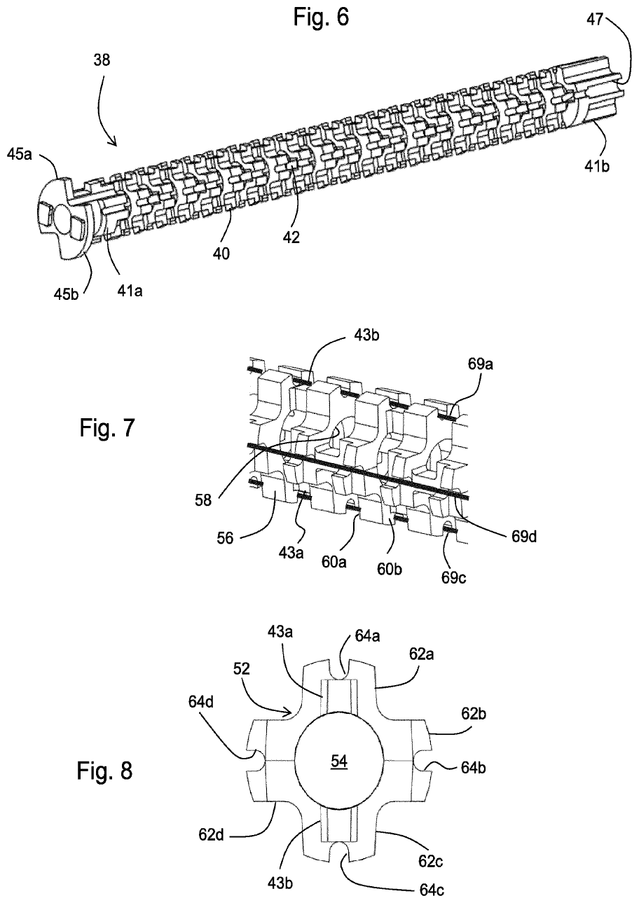

The second part (partly visible in FIG. 3) comprises a spine portion 38 comprising a series of constructional elements which are radially extending spacer segments 40 formed integrally with spine living hinge elements 42, the spine portion being bendable by bending of the spine living hinge elements. The spine portion is discussed in greater detail in relation to FIG. 6, FIG. 7 and FIG. 8.

The interaction between the spine section and the sheath section is also discussed in further detail below in relation to FIG. 9.

FIG. 4 shows a perspective line drawing of a bendable member sheath portion. FIG. 5 shows a close-up view of a part of the same sheath portion. The sheath portion is an integrally moulded polymeric component.

As discussed above, the sheath portion 32 comprising a series of sheath ring segments 34 formed integrally with sheath living hinge elements 36. The sheath portion further comprises two terminal ring segments 35a, 35b, one on either end of the bendable member: a distal terminal ring segment 35a disposed at a distal end of the bendable member and configured for connection to a distal tip assembly on an endoscope, and a proximal terminal ring segment 35b disposed at a proximal end of the bendable member and configured for connection to an adjacent portion of the insertion section.

The sheath ring segments 32 each comprise a radially outer surface 44, a radially inner surface 46, and first and second opposing sidewall surfaces 48a, 48b. Each of the intermediate sheath ring segments (i.e. ring segments other than the terminal ring segments 35a, 35b) are substantially identical to an adjacent ring segment other than for its orientation: each of these selected ring segments is oriented at 90° with an adjacent ring segment when viewed in a cross-section perpendicular to the longitudinal axial direction of the bendable member. A first sidewall surface of a first sheath ring segment is arranged to face a second sidewall surface of an adjacent second sheath ring segment, and the sheath living hinge elements 36 are connected to the sheath ring segments at the first and second opposing sidewall surfaces of adjacent sheath ring segments.

Each sheath living hinge element comprises a pair of flexible web portions 37a, 37b extending from one sidewall surface (e.g. sidewall surface 48b) and extending parallel to the axial direction of the sheath portion. The flexible web portions are disposed to be circumferentially about 180° apart on the sidewall surface. The flexible web portions extend across a gap between axially adjacent sheath ring segments to attach to the axially adjacent sheath ring segment at a sidewall surface (e.g. sidewall surface 48a). The living hinge element (i.e. the pair of flexible web portions 37a, 37b) of each selected sheath ring segment is circumferentially angularly offset with respect to the living hinge element (i.e. the pair of flexible web portions 37a, 37b) of an immediately axially adjacent sheath ring segment by 90°, when viewed in a cross-section perpendicular to the axial direction of the bendable member. The effect of this is that the sheath ring segments of each pair of adjacent sheath ring segments can move relative to each other by bending of the pair of flexible web portions, allowing bending in one of two mutually perpendicular planes for each pair of adjacent sheath ring segments. This arrangement can provide for a suitable controllable yet flexible bending response of the sheath portion.

In the arrangement shown, at least a first living hinge element of the plurality of living hinge elements has a different bending stiffness to a second living hinge element of the plurality of living hinge elements. Specifically, the width of the flexible web portions 37a, b, varies along the length of the sheath portion (width of the webs being measured in a circumferential direction): flexible web portions 37a′ (shown in FIG. 4) and 37b′ (not shown, but oppositely disposed to flexible web portion 37a′) have a smaller width than flexible web portions 37a″ (shown in FIG. 4) and 37b″ (not shown, but oppositely disposed to flexible web portion 37a″). This difference in width results in a variation of bending stiffness between the living hinge element constituted by flexible web portions 37a′ and 37b′, and the living hinge element constituted by flexible web portions 37a″ and 37b″. This can allow for the bending response of the bendable member to be tailored along the length of the bendable member, which may allow for the steering section to be bent with e.g. a more uniform radius of curvature.

In the arrangement shown, the pitch between adjacent living hinge elements is substantially uniform along the length of the sheath portion (pitch being measured as the distance between centres of axially adjacent living hinge elements). The pitch between axially adjacent living hinge elements in this embodiment is about 5 mm.

Each of the sheath ring segments comprises a plurality of chamfered wall portions 50a, b, c, d. The chamfered wall portions are each formed adjacent a living hinge element and are equiangularly spaced about the circumference of the respective sheath ring segment. A first pair of chamfered wall portions 50a, b, is formed between the radially outer surface 44 and the first sidewall surface 48a of the sheath ring segment, and oppositely disposed about a circumference of the sheath ring segment. A second pair of chamfered wall portions 50c, d is formed between the radially outer surface 44 and the second sidewall surface 48b of the sheath ring segment, and oppositely disposed about a circumference of the sheath ring segment. The first and second pairs of chamfered wall portions are oriented at 90° with respect to one another when viewed in a cross-section perpendicular to the longitudinal axial direction of the bendable member. Provision of such arrangements can allow for the sheath portion to achieve smaller bending radii than when one or more such chamfered wall portions are not provided, as impingement of adjacent sheath ring portions on one another during bending can be reduced or avoided.

FIG. 6 shows a perspective line drawing of a bendable member spine portion. FIG. 7 shows a close-up view of a part of the same spine portion. FIG. 8 shows a view from a cross-section of the same spine portion as FIG. 6, the cross section being taken perpendicular to the longitudinal axial direction of the spine portion. The spine portion is an integrally moulded polymeric component.

As discussed above, the spine portion 38 comprises a series of radially extending spacer segments 40 formed integrally with spine living hinge elements 42. The spine portion further comprises two terminal spacer segments 41a, 41b, one on either end of the bendable member: a distal terminal spacer segment 41a disposed at a distal end of the bendable member and configured for connection to a distal tip assembly on an endoscope, and a proximal terminal spacer segment 41b disposed at a proximal end of the bendable member and configured for connection to an adjacent portion of the insertion section.

The distal terminal spacer segment 41a comprises two flange portions 45a, b which are configured to interlock with corresponding recesses 39a, 39b formed in a terminal ring segment 35a of the sheath portion 32 to prevent relative rotational movement of the spine portion and the sheath portion.

The proximal terminal spacer segment 41b includes recesses 47 configured to receive one or more steering wire sheaths (not shown). In this way, when assembled as part of an endoscope, with the spine portion forming a part of the steering section of said endoscope the steering wires can extend through the steering section to the distal tip and the sheaths of the steering wires do not extend through the steering section. Application of tension to the steering wires can therefore cause bending primarily along the length of the steering section, without substantially influencing the curvature of the remaining portion of the insertion section intermediate the hand controller and the steering section.

The spacer segments each comprise a body 52 having a central aperture 54. The central aperture is substantially cylindrical. Each respective central aperture of the series of radially extending spacer segments is substantially aligned to thereby define a spine portion central lumen in which one or more endoscope elements (vacuum tube etc.) can be received when the spine portion is assembled as part of an endoscope.

The spacer segments each comprise a radially outer surface 56, a radially inner surface 58, and first and second opposing sidewall surfaces 60a, 60b. A first sidewall surface of a first spacer segment is arranged to face a second sidewall surface of an axially adjacent second spacer segment. The spine living hinge elements are connected to the spacer segments at the first and second opposing sidewall surfaces and extend in a direction parallel to the longitudinal axis of the spine portion.

Each intermediate spacer segment comprises four spacer arms 62a, b, c, d which extend in a substantially radial direction. These arms are equiangularly spaced about the circumference of the spacer segment body. When the spine portion is used in combination with a sheath, the arms are configured to space at least a part of the radially outer surface of the spacer segment body and the radially inner surface of the sheath to thereby provide one or more longitudinally extending conduits (e.g. conduits 66a, b, c, d as shown in FIG. 9) in the space between adjacent pairs of arms. In the arrangement shown, four longitudinally extending conduits would be provided between the four arms, when the spine portion is coaxially arranged with a sheath. The size of space between adjacent pairs of arms is selected to allow for one or more components of an endoscope, for example a sensor wire, or a fluid conduit, to be arranged in said space.

Each of the four spacer arms 62a, b, c, d comprises a single slot 64a, b, c, d formed at a radially outer extent of the arm. These slots are suitably sized to each receive a steering wire 69a, b, c, d, and to thereby act as a guide for said wire.

Each spine living hinge element 42 comprises a pair of flexible web portions 43a, 43b extending from one sidewall surface (e.g. sidewall surface 60b) and extending parallel to the axial direction of the spine portion. The flexible web portions are disposed to be circumferentially about 180° apart on the sidewall surface. The flexible web portions extend across a gap between axially adjacent spacer segments to attach to the axially adjacent spacer segment at a sidewall surface (e.g. sidewall surface 60a). The living hinge element (i.e. the pair of flexible web portions 43a, 43b) of each selected spacer segment is circumferentially angularly offset with respect to the living hinge element (i.e. the pair of flexible web portions 43a, 43b) of an immediately axially adjacent spacer segment by 90°, when viewed in a cross-section perpendicular to the axial direction of the bendable member. The effect of this is that the sheath ring segments of each pair of adjacent sheath ring segments can move relative to each other by bending of the pair of flexible web portions, allowing bending in one of two mutually perpendicular planes for each pair of adjacent sheath ring segments. This arrangement can provide for a suitable controllable yet flexible bending response of the spine portion.

In the arrangement shown, when the steering section is straight, at least a first spine living hinge element of the plurality of spine living hinge elements has a different bending stiffness to a second living hinge element of the plurality of living hinge elements. This is provided for by varying the width of the flexible web portions constituting the living hinge elements along the length of the spine portion, in a similar manner as described above in relation to the sheath portion.

In the arrangement shown, the pitch between adjacent living hinge elements is substantially uniform along the length of the spine portion (pitch being measured as the distance between centres of adjacent living hinge elements). The pitch between adjacent living hinge elements is about 5 mm.

FIG. 9 shows a cross-sectional view of the same part of an endoscope insertion section as shown in FIG. 2, the cross section being taken perpendicular to the axial direction of the insertion section. In this view, it can be seen that the spine portion and sheath portion are coaxially arranged to thereby define multiple longitudinally extending conduits 66a, b, c, d and 68a, b, c, d between the spacer segments of the spine portion and the sheath ring segments of the sheath portion. More specifically, these conduits are defined between the radially outer surface of the spacer segment body and the radially inner surface of the sheath portion. All conduits are equiangularly spaced about the bendable member.

A first set of conduits 66a, b, c, d is defined between each adjacent pairs of spacer arms 62a, b, c, d of the spine portion 38 and the radially inner surface of the sheath portion 32. Each of these conduits may be designated for use in provided one or more functions of an endoscope, for example designated for use to conduct a flow of irrigation liquid to the distal tip, to conduct a flow of insufflation gas to the distal tip, or to conduct one or more electrical conductors to provide power to the distal tip.

A second set of conduits 68a, b, c, d is defined by the slots 64a, b, c, d formed at a radially outer extent of the spacer arms 62a, b, c, d and the radially inner surface of the sheath portion 32. Each of these conduits is configured to receive a steering wire 69a, b, c, d such that the conduits can each act as a steering wire guide.

Finally, it can also be seen that the flexible webs 43a, b constituting the spine living hinge elements and the flexible webs 37a, b constituting the sheath living hinge elements are substantially circumferentially and axially aligned, thereby allowing for bending of the bendable member to be facilitated.

The bendable member is assembled by providing the spine portion 38, optionally arranging one or more endoscope components on and/or in said spine portion, providing the sheath portion 32, and coaxially arranging the spine portion 38 and sheath portion 32 such that the longitudinally extending conduits are defined between the spacer segments of the spine portion and the sheath ring segments of the sheath portion (e.g. by rotationally aligning the spine portion and the sheath portion to a predetermined relative rotational arrangement). One or more further endoscope assembly steps may then be performed in a known manner. Alternatively, the bendable member may be supplied as a standalone unit to third parties for subsequent incorporation in e.g. an endoscope.

The second embodiment of the present invention (shown in FIG. 10-13) has a generally similar overall structure to the first embodiment, discussed above. Accordingly, where features of this embodiment are not explicitly discussed, it should be assumed that the above description of the equivalent features of the first embodiments applies.

FIG. 10 shows a perspective line drawing of part of an endoscope insertion section incorporating a bendable member according to this second embodiment. The insertion section includes a steering section 16 and a distal tip assembly 18. Also visible is a part of the insertion section intermediate the proximal end of the insertion section (the end connected to the hand controller, not shown) and the steering section—referred to herein as an insertion section main body 17. It can be seen that in this embodiment, both the steering section 16 and the insertion section main body 17 respectively comprise different bendable members comprising a series of constructional elements arranged longitudinally in series along the bendable member and formed integrally with a plurality of living hinge elements that permit bending between adjacent constructional elements.

Whilst not shown in detail, the insertion section main body 17 comprises multiple bendable members (specifically three) connected in series along the length of the insertion section main body. Each of these bendable members is a moulded polymeric component comprising a series of constructional elements which are ring segments 134 formed integrally with living hinge elements 136, each of the bendable members being bendable by bending of the sheath living hinge elements. The end of the insertion section main body proximal the hand controller is configured to have a higher bending stiffness (i.e. a higher resistance to bending) than a portion of the insertion section main body distal to the hand controller. This is achieved by providing a first set of living hinge elements having a first thickness at the end of the insertion section main body proximal the hand controller, and a second set of living hinge elements having a second thickness in the portion of the insertion section distal to the hand controller, wherein the first thickness is greater than the second thickness. In other words, by providing thicker living hinge elements at the end of the insertion section main body proximal the hand controller as compared with the living hinge elements in the portion of the insertion section distal to the hand controller. Specifically, the radial thickness of each of the pairs of flexible web portions 137a′, 137b′, 137c′ (the first set proximal the hand controller) is larger than the radial thickness of each the pairs of flexible web portions 137a″, 137b″, 137c″ (the second set distal to the hand controller). This difference in thickness results in a variation of bending stiffness between the living hinge elements comprising flexible web portions 137a′, 137b′, 137c′ and the living hinge element comprising flexible web portions 137a″, 137b″, 137c″ and accordingly results in an increase in torsional stiffness of the insertion section main body proximal to the hand controller as compared with the torsional stiffness of the insertion section main body distal to the hand controller, thereby allowing an operator to more easily transmit torque from the hand controller to the insertion section main body.

The bendable members forming part of the insertion section main body are substantially cylindrical, and do not include a spine portion of the type used in the steering section. Other endoscope components (vacuum tube, steering wires etc.) are therefore unconstrained within the central aperture of the insertion section main body. Such an arrangement can reduce the construction costs of the device.

Turning to review the steering section of this embodiment more closely, it can be seen that similarly to the first embodiment, the steering section 16 includes a bendable member comprising first and second coaxially arranged parts, each of the coaxially arranged parts being formed from a series of constructional elements arranged longitudinally in series along the bendable member and formed integrally with a plurality of living hinge elements that permit bending between adjacent constructional elements.

The first part comprises a sheath portion 32 comprising a series of constructional elements which are sheath ring segments 34 formed integrally with sheath living hinge elements 36, the sheath portion being bendable by bending of the sheath living hinge elements. FIG. 11 shows a close-up view of a part of the sheath portion. The sheath portion is an integrally moulded polymeric component.

The sheath portion 32 of the second embodiment differs from that of the first embodiment in that the chamfered wall portions are larger than those of the first embodiment. It can be seen from FIG. 11 that each of the sheath ring segments comprises a plurality of chamfered wall portions—three of these are indicated by reference FIGS. 70a, b, c in FIG. 11. A first pair of chamfered wall portions 70a, b, is formed between a radially outer surface 72 and a first sidewall surface 74a of the sheath ring segment, and oppositely disposed about a circumference of the sheath ring segment. A second pair of chamfered wall portions (the pair comprising portion 70c, as well as a further portion not visible in FIG. 11), is formed between the radially outer surface 72 and a second sidewall surface 74b of the sheath ring segment, and oppositely disposed about a circumference of the sheath ring segment. The first and second pairs of chamfered wall portions are oriented at 90° with respect to one another when viewed in a cross-section perpendicular to the axial direction of the bendable member.

Each of these chamfered wall portions extends angularly for more than 90° around the circumference of the sheath ring segment—for example, for a circumferential length that is greater than 0.25 times the circumferential length of a sheath ring segment. In this way, parts of the first pair of chamfered wall portions and the second pair of chamfered wall portions overlap in a circumferential direction about the circumference of the sheath ring segment (albeit on opposite sides of the sheath ring segment). This is in comparison to the first embodiment, where the first pair of chamfered wall portions 50a, b and the second pair of chamfered wall portions 50c, d, on each sheath ring portion do not overlap in a circumferential direction (see FIG. 5). This arrangement has been found to be particularly advantageous as it can allow enhanced flexibility of the bending member during bending. Furthermore, where an outer casing is provided, the larger gaps between sheath ring segments provided by the larger chamfers can allow the outer casing to deflect into the chamfered areas without restricting the tight curl of the whole assembly.

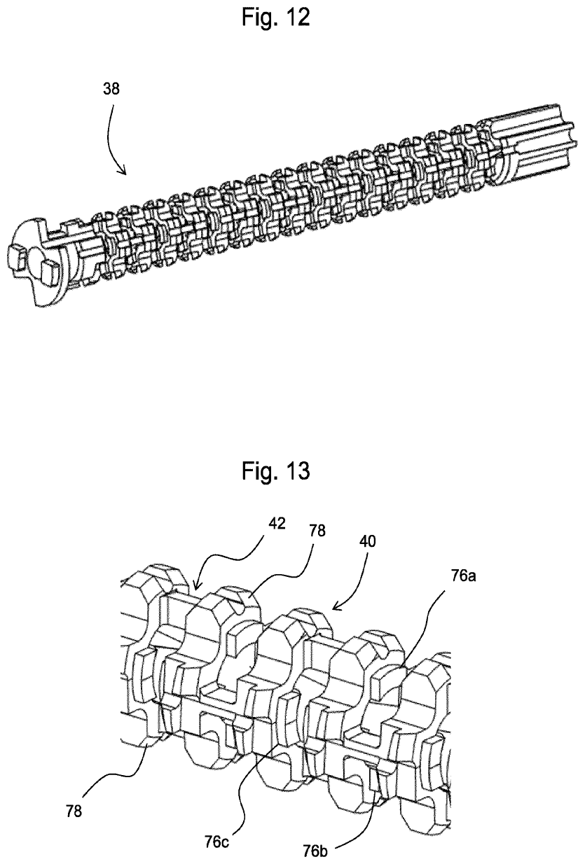

The second part (partly visible in FIG. 10, and shown in detail in FIGS. 12 and 13) comprises a spine portion 38 comprising a series of constructional elements which are radially extending spacer segments 40 formed integrally with spine living hinge elements 42, the spine portion being bendable by bending of the spine living hinge elements. FIG. 13 shows a close-up view of a part of the spine portion. The spine portion is an integrally moulded polymeric component.

The spine portion 40 of the second embodiment differs from that of the first embodiment in a number of ways. Firstly, each spacer segment comprises two pairs of projections disposed on its sidewall surfaces: a first pair of projections 76a, b formed on a first sidewall surface, and a second pair of projections disposed on a second sidewall surface (projection 76c visible, the second of this pair of projection not visible in FIG. 13). These projections reduce the open spacing between adjacent spacer segments. This can help to reduce the risk of any components or tools located within the central aperture of the spacer segment from getting caught in the gaps between spacer segments, thus improving both safety and ease of use of e.g. an endoscope incorporating the bendable member.

Furthermore, the spacer arms are configured to have a more ‘rounded’ profile than those of the first embodiment, by provision of chamfers 78 formed between a radially outer surface of the spacer segment at the tip of each arm, and the first and second sidewall surfaces of the spacer segment. In other words, the spacer arms are chamfered. This arrangement can allow the spine portion to achieve a smaller/tighter bending radius than when one or more such chamfers are not provided, as impingement of spacer arms on adjacent spacer segments during bending can be reduced or avoided.

Other features of the spine portion are generally similar as the first embodiment. Furthermore, the spine portion and the sheath portion interact in substantially the same manner as in the first embodiment, as shown and discussed in relation to FIG. 9.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilized for realizing the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A bendable member for an imaging endoscope, said bendable member comprising a series of constructional elements arranged longitudinally in series along the bendable member and formed integrally with a plurality of living hinge elements that permit bending between adjacent constructional elements, wherein, when the bendable member is straight, a first living hinge element of the plurality of living hinge elements has a different bending stiffness to a second living hinge element of the plurality of living hinge elements.

2. The bendable member according to claim 1 wherein a first set of living hinge elements have a first predetermined bending stiffness, and a second set of living hinge elements have a second predetermined bending stiffness, and wherein the first and second predetermined bending stiffnesses are different.

3. The bendable member according to claim 1 wherein the bending stiffness of each of the living hinge elements along the length of the bendable member differ from one another.

4. The bendable member according to claim 1 wherein the first living hinge element has at least one dimension which is different to a corresponding dimension of the second living hinge element.

5. The bendable member according to claim 1 wherein the living hinge elements comprise a pair of flexible web portions.

6. The bendable member according to claim 5 wherein at least one of (i), (ii) and (iii) applies:
(i) the axial length of the flexible webs of the first living hinge element is different to the length of the flexible webs of the second living hinge element.
(ii) the circumferential width of the flexible webs of the first living hinge element is different to the width of the flexible webs of the second living hinge element.
(iii) the radial thickness of the flexible webs of the first living hinge element is different to the width of the flexible webs of the second living hinge element.

7. The bendable member according to claim 1 wherein the pitch between adjacent living hinge elements is substantially uniform along the length of the bendable member, optionally wherein the pitch between adjacent living hinge elements is in a range of from 1 mm to 20 mm.

8. The bendable member according to claim 1 wherein each selected living hinge element is circumferentially angularly offset with respect to an immediately axially adjacent living hinge element by 90° when viewed in a cross-section perpendicular to the axial direction of the bendable member.

9. The bendable member according to claim 1 wherein the constructional elements comprise a body having at least a radially outer surface, and first and second opposing sidewall surfaces, and wherein each living hinge element comprises a pair of flexible web portions extending parallel to the axial direction of the bendable member from the first sidewall surface of a first constructional element to the second sidewall surface of an axially adjacent constructional element, wherein the flexible web portions are disposed to be circumferentially about 180° apart on the sidewall surface of the constructional elements.

10. The bendable member according to claim 1 wherein the constructional elements comprise a body having at least a radially outer surface, and first and second opposing sidewall surfaces, and wherein a chamfered wall portion is formed between the radially outer surface and at least one of the first and second sidewall surfaces.

11. The bendable member according to claim 1 wherein the bendable member comprises first and second coaxially arranged parts, each of the coaxially arranged parts of the bendable member being formed from a series of constructional elements arranged longitudinally in series along the bendable member and formed integrally with a plurality of living hinge elements that permit bending between adjacent constructional elements.

12. The bendable member according to claim 11 wherein:
the first coaxially arranged part comprises a sheath portion comprising a series of sheath ring segments formed integrally with sheath living hinge elements, the sheath portion being bendable by bending of the sheath living hinge elements; and
the second coaxially arranged part comprises a spine portion comprising a series of radially extending spacer segments formed integrally with spine living hinge elements, the spine portion being bendable by bending of the spine living hinge elements; wherein one or both of (i) and (ii) applies:
(i) a first sheath living hinge element has a different bending stiffness to a second sheath living hinge element;
(ii) a first spine living hinge element has a different bending stiffness to a second spine living hinge element.

13. An imaging endoscope comprising:
a hand controller;
an insertion section having a proximal end connected to the hand controller and a distal end for insertion into a subject, wherein a distal tip assembly is located at the distal end of the insertion section and a steering section is located adjacent and proximal to the distal tip, said steering section being bendable for steering by operation of the hand controller;
wherein at least a part of the insertion section comprises a bendable member, said bendable member comprising a series of constructional elements arranged longitudinally in series along the bendable member and formed integrally with a plurality of living hinge elements that permit bending between adjacent constructional elements, wherein, when the bendable member is straight, a first living hinge element of the plurality of living hinge elements has a different bending stiffness to a second living hinge element of the plurality of living hinge elements.

14. The imaging endoscope according to claim 13 wherein the part of the insertion section comprising the bendable member includes the steering section.

15. The imaging endoscope according to claim 13 wherein the part of the insertion section comprising the bendable member includes a part of the insertion section intermediate the proximal end of the insertion section and the steering section.
